(19) United States
(12) Patent Application Publication
(10) Pub. No.: US 2019/0343853 A1
(43) Pub. Date: Nov. 14, 2019

(54) METHODS OF TREATING HYPERTENSION

(71) Applicant: THERACOS SUB, LLC, Marlborough, MA (US)

(72) Inventors: Jordan MECHANIC, San Jose, CA (US); Brian SEED, Boston, MA (US)

(21) Appl. No.: 16/393,755

(22) Filed: Apr. 24, 2019

Related U.S. Application Data
(60) Provisional application No. 62/662,526, filed on Apr. 25, 2018.

(51) Int. Cl. A61K 31/70; A61K 45/06

(52) U.S. Cl. CPC A61K 31/70; A61K 31/4433; A61K 45/06

(57) ABSTRACT

Provided are methods for treating primary (or essential) hypertension in a subject, said method comprising administering to a subject in need thereof a therapeutically effective amount of Compound 1, having the formula

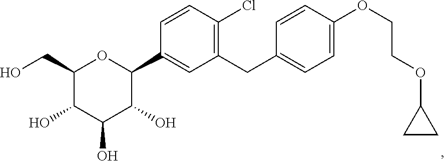 [SMILES: OC[C@H]1O[C@@H](c2ccc(Cl)c(Cc3ccc(OCCOC4CC4)cc3)c2)[C@H](O)[C@@H](O)[C@@H]1O]

or a pharmaceutically acceptable form thereof.

### CROSS-REFERENCES TO RELATED APPLICATIONS

This application claims the benefit of priority under 35 U.S.C. § 119(e) to U.S. Provisional Application Ser. No. 62/662,526, filed Apr. 25, 2018, the disclosure of which is incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Hypertension, or high blood pressure, affects about 50 million Americans and is a major risk factor for coronary heart disease, stroke, and heart failure (Centers for Disease Control and Prevention and National Institutes of Health “Heart Disease and Stroke” Healthy People 2010-Conference Edition, November 1999). Approximately 90-95% of all hypertension develops from unknown causes. This form of hypertension is often referred to as primary (or essential) hypertension to distinguish it from secondary hypertension that arises from known cause (i.e., due to specific illness or medical treatment) (Kaplan 1998).

Although the etiology of primary hypertension is unknown, it is unlikely that there is a single causative basis for the disorder. There is strong evidence of a hereditary predisposition for hypertension as well as for the involvement of environmental factors such as dietary sodium intake, obesity, stress and dyslipidemia with its consequent atherosclerosis.

Numerous medications have been developed for the treatment of hypertension. Among the most commonly used classes of therapeutic agent are angiotensin converting enzyme (ACE) inhibitors and angiotensin II receptor blockers (ARBs), cardiac calcium (L-channel) blockers, agents that decrease sympathetic tone, such as α- and β-adrenergic receptor blockers (α- and β-blockers), diuretics of three general classes (thiazides, loop diuretics, and epithelial sodium channel (ENaC) inhibitors), aldosterone antagonists and renin inhibitors. The diversity of mechanisms that can be defeated to functionally improve vascular state reflects the complexity and redundancy of the physiological pathways that control blood pressure.

Hypertension can be characterized by an increase in the total peripheral vascular resistance or an increase in cardiac output, or both. In high-output hypertension, peripheral intrinsic resistance is low but blood pressure is elevated because the total blood flow is high, and the frictional resistance to flow is proportional to velocity. This form of hypertension is often associated with increased adrenergic tone and dysregulation of calcium homeostasis. Forms of hypertension characterized by peripheral vascular resistance often occur in the context of normal or reduced cardiac output and have been attributed to vasoreactivity and/or fluid retention that results from homeostatic preservation of osmotic pressure coupled with increased sodium retention through renal mechanisms.

The kidneys play an important role in maintaining blood pressure. The basic anatomical unit of the kidney is the nephron, the tubular path through which fluid flows from plasma to urine. Each nephron begins with a glomerulus, which is a tuft of capillaries and specialized endothelium that leaks plasma into the nephron. From there the fluid passes through a single channel characterized by anatomically and functionally distinct sequential segments, culminating in a collecting duct formed by the merger of multiple nephrons.

On average, for a typical healthy human approximately 180 L of plasma containing 1.6 kg of NaCl passes from the glomeruli into the renal proximal tubules each day. From this input the kidneys reabsorb all but approximately 2 L of water and 3.5 g of NaCl. The precise amounts of unabsorbed water and NaCl are affected by many factors, among which diet is the most important in most settings. With very little variation the total sodium in diet must be balanced by the total sodium in excreta, to which urine typically contributes the greatest proportion. The remarkable balancing that the kidney performs is a consequence of a network of feedback mechanisms that couple blood pressure in the glomeruli to fluid flow in the distal convoluted tubules, each of which rejoins its own glomerulus in a structure called the macula densa. Tubulo-glomerular feedback in the macula densa causes blood pressure to increase when tubular fluid flux is low, and to decrease when fluid flux is high, thereby ensuring a nearly constant rate of flow.

Tubulo-glomerular feedback is based on sensing of the NaCl concentration in the tubular fluid by a mechanism that relies on a plasma membrane protein that cotransports one Na⁺ ion, one K⁺ ion and two Cl⁻ ions as a functional unit. The NaCl concentration is low when fluid flux is low because a greater proportion of NaCl can be removed when the fluid passes slowly through the upstream tubules. In turn, low NaCl concentration causes the cells of the macula densa to synthesize prostaglandins, which cause juxtaglomerular cells to release renin, an enzyme that promotes increased blood pressure through the renin-angiotensin-aldosterone system (RAAS). By a different mechanism involving release of ATP, the arterioles supplying the glomeruli constrict when the NaCl concentration/fluid flux is high, reducing plasma flow to the glomeruli. Thus, both local/prompt and systemic/deferred mechanisms help sustain a consistent flow.

The role of the kidney is not limited to reuptake of water and NaCl. Many important metabolites in plasma are also recovered from the tubular fluid by an active transport process. In this way the kidney discards nearly the entire contents of the plasma, then selectively retrieves the components that are necessary for survival. This apparently inefficient design has great power to protect the body against environmental toxins, the vast majority of which cannot be predicted by any consistent mechanism. By adopting a default discard design, the kidney can ensure that a vast universe of poisons need not be detected to be excreted.

Glucose, as the principal energy source for many of the cells in the body, is an especially important metabolite to be retrieved from the filtered plasma. The kidneys recover glucose from filtrate by a two-stage process of active transport involving two sodium-glucose linked transporter (SGLT) proteins, called SGLT1 and SGLT2. As their name suggests, these transporters allow glucose to be taken up from the lumen of the tubule by a process that exploits the transmembrane potential for sodium ions, which have a higher concentration in the extracellular medium than in the cytoplasm. By coupling sodium and glucose transport, the SGLT proteins allow dilute glucose outside the cell to be concentrated in the cells lining the initial segments of the renal proximal tubules. The concentrated glucose then passively diffuses back into the circulation through basolateral equilibrative transporters that allow glucose to move out of or into the cell in whichever direction has lower concentration.

SGLT2 is a high capacity, low affinity transporter that is nearly exclusively expressed in the kidney in the first two segments of the proximal tubule, whereas SGLT1 is a lower capacity higher affinity transporter expressed in both the small intestine and the second two segments of the proximal tubule. SGLT2 cotransports one molecule of glucose with one Na⁺ ion whereas SGLT2 cotransports one molecule of glucose with two Na⁺ ions. The tandem anatomical arrangement of the transporters allows most of the glucose to be recovered through the energetically more efficient SGLT2 transporter, with the remainder to be retrieved by the energetically less efficient SGLT1 transporter.

Because the recovery of glucose requires internalization of Na⁺ ions, blockade of SGLT2 causes both glucose and Na⁺ to remain outside the cell. To some extent the Na⁺ and glucose that remain can then be imported via SGLT1, but the practical consequence is that lumenal glucose is incompletely absorbed. The resulting glucose can contribute to total fluid excretion by a mechanism called osmotic diuresis, which signifies that the osmotic pressure conferred by an unabsorbable solute prevents concentration of the urine that otherwise might have taken place.

Different forms of hypertension can respond to a greater or lesser degree to the pharmacological or lifestyle intervention intended to palliate the course of disease. For example, the blood pressure of some individuals is highly responsive to the amount of salt in the diet, whereas the pressure in salt-resistant individuals does not show a similar dependence. The consequences of administration of peripheral vasodilators in high-output hypertension are relatively limited compared to the effects of agents that decrease cardiac output, such as 3-blockers and calcium channel blockers. As the disease progresses it is common to find that affected individuals require multiple anti-hypertensive medications, each addressing a different mechanism, to control their disease.

Administration of SGLT2 inhibitors to treat type 2 diabetes mellitus (T2DM) has become an accepted medical strategy that has in at least some cases provided cardiovascular benefits by a mechanism that is incompletely understood. Reduction in blood pressure has been observed in some but not all studies of SGLT2 inhibitors for T2DM, and it remains unclear to what degree the vascular effects can be attributable to the higher blood glucose burden of diabetic individuals and the associated higher renal glucosuria in the context of SGLT2 inhibition. The glucosuria of diabetics treated with SGLT2 inhibitors can be profound, and up to double the glucosuria observed in healthy volunteers.

Several studies have been conducted of the effects of SGLT2 inhibitors on blood pressure in individuals with diabetes, and blood pressure data have been collected in many trials measuring SGLT2 inhibitor effects on glycemic control. The nexus between diabetes and hypertension is medically important because a significant proportion of both hypertensives and diabetics are affected by both diseases, and the prevalence of co-morbidity increases with age and disease severity.

Blood pressure measurements can be conducted with the help of one or more devices designed for the purpose, called sphygmomanometers. Two measurements are usually recorded, the pressure corresponding to the peak arterial pressure and resulting from the contraction of the ventricles of the heart (systole), and the pressure minimum detected when the ventricles are filling (diastole). The difference between the systolic blood pressure (SBP) and diastolic blood pressure (DBP) is the pulse pressure. Ordinarily blood pressure and heart rate are coordinated to sustain the blood flow required by systemic oxygen demand, so that the heart beats more rapidly when blood pressure is low, and more infrequently when blood pressure is high.

Detection of blood pressure is based on the auscultation of an appropriate artery by a sound detection device, such as a stethoscope in manual determinations, or a microphone in automated determinations. A cuff supplying pressure to an appropriate anatomical site, usually in the upper arm (brachium) but sometimes in the wrist or ankle, is fastened about the limb and inflated above the pressure needed to completely collapse the arteries, allowing no blood to pass. The cuff is then gradually deflated and the arteries auscultated for two critical sound transitions: the first detection of blood spurting through the constriction as the cuff pressure ceases to completely occlude the arteries, for which the corresponding pressure is taken to be the systolic blood pressure; and the final loss of sound corresponding to the fluid spurting through the constriction, for which the corresponding pressure is taken to be the diastolic blood pressure. The units of measure are millimeters of mercury, derived from the construction of early sphygmomanometers that measured pressure by the displacement of a column of mercury in a graduated sealed tube.

Blood pressure measurements can vary widely as the blood pressure itself is highly dynamic and closely follows the metabolic needs posed by activity and environmental influences. For a resting seated office measurement, a systolic blood pressure of 120 mm Hg and a diastolic pressure of 80 mm Hg are considered normotensive. One such reading would typically be recorded in abbreviated form as 120/80. Measurements of 140/90 mm Hg or above are considered to indicate hypertension, although a recent guideline has proposed that the lower limit be reduced.

A variety of automated instruments for recording blood pressure can be used for the measurement. Among the more important for clinical studies are those that produce a record of multiple determinations throughout the day by a technique called ambulatory blood pressure monitoring (ABPM.)

For the latter, the subject wears a battery-powered sphygmomanometer throughout the course of the monitoring period (typically 24 h) and measurements are taken on a regular basis by automated inflation and relaxation of the cuff. The recorded measures are retained and subsequently transferred to a database for analysis. ABPM is considered to produce more consistent and reliable data sets for hypertension studies, as the data are less confounded by effects that are known to influence blood pressure, such as diurnal variation and office visit-specific hypertension (sometimes called “white coat hypertension”). The latter is thought to be provoked in some individuals by the stress of an office visit.

Blood pressure is subject to homeostatic regulation depending on posture, and healthy individuals rarely sense differences in blood pressure on rising from a sitting or supine position. However in some individuals and in certain circumstances the circulatory system fails to compensate appropriately rapidly and a characteristic feeling sometimes described as “light-headed” or “dizzy” and associated with insufficient delivery of blood to the brain may ensue. In severe cases a loss of consciousness may occur. This transient effect is called postural or orthostatic hypotension and can be an undesired side effect of antihypertensive medications. To avoid making comparisons between different postural states, most studies of hypertension that entail only office or home (self-directed) measurements often specify a particular postural state, usually the seated state. To assess orthostatic hypotension, a measurement is usually made immediately after an individual is asked to stand after lying in a supine pose for at least five minutes.

Sustained hypotension can have deleterious consequences, including loss of consciousness that can be fatal or contribute to fatal events, such as falls or motor vehicle accidents. Blood pressure medications that have lower risk of generating hypotensive states are therefore preferred therapeutically.

To date no studies of the effects of SGLT2 inhibitors on blood pressure in individuals that do not exhibit diabetes have been announced or reported. Among studies of diabetics conducted with a blood pressure endpoint, Weber et al. 2016 Lancet Diabetes Endocrinol 4: 211 studied the effects of dapagliflozin in a population with uncontrolled diabetes that was also afflicted by hypertension. The co-primary endpoints were changes from baseline to week 12 in office seated systolic blood pressure and in HbA_{1c}.

HbA_{1c} is a form of hemoglobin in which the N-terminal valine residues have undergone non-enzymatic addition of glucose, a process that occurs naturally with a rate that is dependent on the concentration of glucose in the blood. Because human erythrocytes have a lifetime of approximately 90 days, the percent of hemoglobin that has undergone non-enzymatic glycation reflects the average glucose concentration over the lifespan of the erythrocyte. The placebo-corrected reduction in HbA_{1c}, is a well-accepted surrogate endpoint for the assessment of glycemic control in diabetes trials.

Weber et al. reported that for the 10 mg dapagliflozin dose, which was the more effective, the placebo-corrected change in HbA1c was −0.61%, and the placebo-adjusted change from baseline in seated office systolic blood pressure was −4.28 mm Hg. A secondary endpoint was the placebo-corrected change in systolic blood pressure from baseline to week 12, as assessed by ABPM. A decrease in SBP of 4.45 mm Hg was observed by this metric.

In a meta-analysis of SGLT2 inhibitor effects on blood pressure Baker et al. 2014 J Am Soc Hypertens: JASH 8:262 concluded that the treatment effect of SGLT2 inhibitors was responsible for a reduction in SBP from baseline of 4.0 mm Hg and a reduction in DBP from baseline of 1.6 mm Hg, and that SGLT2 inhibitor exposure did not increase the incidence of orthostatic hypotension. The SGLT2 inhibitors studied were canagliflozin, dapagliflozin, empagliflozin, ipragliflozin and remogliflozin, the last of which was never approved in any country.

In a meta-analysis of ABPM outcomes reported for trials of SGLT2 inhibitors in diabetic populations, Baker et al. 2017 J Am Heart Assoc. 6: e005686 reported that SGLT2 inhibitors as a class have been associated with reductions in SBP of −3.76 mm Hg (95% confidence interval, −4.23 to −2.34); and with reductions in DBP of −1.83 mm Hg (95% CI, −2.35 to −1.31. The reductions were not limited to a specific time of day and an association with baseline BP or degree of decrease in body weight (factors with the power to potentially contribute to the measured effects) was not observed.

Meta-analyses are considered to be useful tools for assessing class effects, the effects that can be attributed to the primary or secondary pharmacological influences of the class of compounds studied. Class effects necessarily do not take into account potentially important functional distinctions between drugs within a class, that may result from differences in potency, pharmacokinetics, distribution, metabolism or side effect profiles, among many factors.

Of all the potential confounding factors to be considered in studies of the effects of SGLT2 inhibition on hypertension, the largest is likely contributed by the population itself. Amelioration of diabetes by a variety of interventions mechanistically unrelated to SGLT2 inhibition has been shown to produce reductions in blood pressure in diabetic hypertensives. For example, use of the GLP-1 receptor agonists exenatide and liraglutide have produced reductions in SBP of between 4.5 and 5.6 mm Hg relative to placebo (Wong 2013 Diab Obes Metab 15:737) and a meta-analysis of thiazolidinedione effects has shown a mean reduction of SBP of 7.7 mm Hg across 37 randomized clinical trials (Qayyum 2006 J Clin Hypertens 8:19-28). Smaller reductions have been reported to attend the use of DPP-4 inhibitors and metformin (Baker et al. 2014). Hence treatment of diabetes may intrinsically result in improvement in hypertension that is clinically meaningful. Although variations in the efficacy of drugs of different classes is apparent, in the absence of detailed study in non-diabetic populations it cannot be excluded that the apparently less effective agents produce exacerbations of hypertension counterbalanced by the palliative effect of improved glycemic control. Thus the degree to which an antidiabetic agent intrinsically reduces blood pressure must be studied in a population unselected for hyperglycemia. This requirement is especially important when the mechanism of action is related to diuresis and natriuresis, the magnitude of which is increased in the diabetic state, as appears to be the case for the SGLT2 inhibitors as a class.

Because SGLT2 inhibitors have favorable safety profiles, and in particular have negligible risk of causing hypoglycemia in euglycemic individuals, or in diabetic individuals managed solely by SGLT2 inhibition alone, they represent a potentially attractive new approach to the treatment of essential hypertension.

Despite the number of antihypertensive agents available, the percent of individuals with hypertension for which the disease is inadequately controlled by lifestyle change and medication is nearly 50% in the United States and the incidence of hypertension is increasing.

As such, there is a need in the art for identifying additional therapy options in treating primary hypertension. The present disclosure addresses this need and provides related advantages as well.

### BRIEF SUMMARY OF THE INVENTION

Provided herein are methods of treating primary (or essential) hypertension in a subject. The methods involve treating the subject with Compound 1, having the formula:

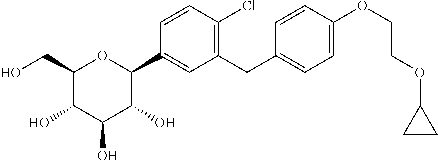 [SMILES: OC[C@H]1O[C@@H](c2ccc(Cl)c(Cc3ccc(OCCOC4CC4)cc3)c2)[C@H](O)[C@@H](O)[C@@H]1O]

or a proline complex thereof, or a bis-proline complex thereof. The compound is known to have an inhibitory effect on sodium-dependent glucose cotransporters (SGLT), particularly sodium-dependent glucose cotransporter 2 (SGLT2); however, its usefulness in reducing primary hypertension was previously unknown. Also provided herein are pharmaceutical compositions, independently or in combination with other therapeutic agents, for treating diseases and conditions which are associated with primary hypertension.

### BRIEF DESCRIPTION OF THE DRAWINGS

FIG. 1 provides the X-ray powder diffraction (XRPD) spectra of crystalline (2 S,3R,4R,5 S,6R)-2-(4-chloro-3-(4-(2-cyclopropoxyethoxy)benzyl)phenyl)-6-(hydroxymethyl)tetrahydro-2H-pyran-3,4,5-triol.

FIG. 2 provides a Table of XRPD data for the XRPD spectra in FIG. 1.

FIG. 3 provides the Raman spectra of crystalline (2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol.

FIG. 4 provides a Raman peak list for the Raman spectra in FIG. 3.

FIG. 5 shows the change in seated office systolic blood pressure as a function of time, in subjects administered EGT0001442 (Compound 1) as compared to a placebo group.

FIG. 6 shows the change in seated office diastolic blood pressure as a function of time, in subjects administered EGT001442 (Compound 1) as compared to a placebo group.

FIG. 7 shows the effects of Compound 1 on the change in systolic and diastolic blood pressure as a function of time among subjects who had a systolic blood pressure of 140 mm Hg or above at study entry.

### DETAILED DESCRIPTION OF THE INVENTION

Described herein are methods for treating primary (or essential) hypertension in a subject. The methods involve treating the subject with Compound 1, having the formula:

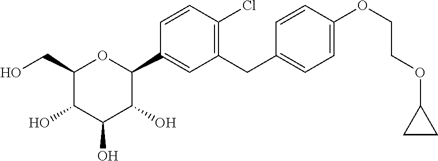 [SMILES: OC[C@H]1O[C@@H](c2ccc(Cl)c(Cc3ccc(OCCOC4CC4)cc3)c2)[C@H](O)[C@@H](O)[C@@H]1O]

or a proline complex thereof, or a bis-proline complex thereof. The compound is known to have an inhibitory effect on sodium-dependent glucose cotransporters (SGLT), particularly sodium-dependent glucose cotransporter 2 (SGLT2). The methods include specific dosing regimens that have significant efficacy in treating the subject and that are well tolerated in subjects. As a result of SGLT2 inhibition, the renal tubular fluid is expected to contain elevated concentrations of both glucose and sodium ions. A mild diuretic effect is both predicted and empirically observed in human populations administered SGLT2 inhibitors. The combined effects of natriuresis and diuresis may account for the experimentally observed protective effects of Compound 1 in the spontaneously hypertensive rat, stroke prone strain (SHRSP) that have previously been reported (Zhang 2011). However, because of differences in receptor specificity and other physiologic differences, it is not clear that a similar action could be observed in human populations affected by essential hypertension.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

As used herein, the terms “a,” “an,” or “the”, not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

As used herein, the terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

Any ranges used herein, for example “from 5 to 100” are meant to include both endpoints of the stated range, as well as all intermediate ranges even though not specifically stated. The range “from 5 to 100” also includes, for example “5 to 90”, “10 to 100”, “22 to 32” and the like.

As used herein, “treatment” or “treating,” or “palliating” or “ameliorating” are used interchangeably herein. These terms refer to an approach for obtaining beneficial or desired results including but not limited to a therapeutic benefit. By therapeutic benefit is meant amelioration of the underlying disorder being treated. Also, a therapeutic benefit is achieved with the amelioration of one or more of the physiological symptoms associated with the underlying disorder such that an improvement is observed in the patient, notwithstanding that the patient may still be afflicted with the underlying disorder. Treatment includes causing the clinical symptoms of the disease to slow in development by administration of a composition; suppressing the disease, that is, causing a reduction in the clinical symptoms of the disease; inhibiting the disease, that is, arresting the development of clinical symptoms by administration of a composition after the initial appearance of symptoms; and/or relieving the disease, that is, causing the regression of clinical symptoms by administration of a composition after their initial appearance. For example, certain methods described herein treat primary hypertension by decreasing or reducing the occurrence or progression of high blood pressure.

As used herein, the term “effective amount” or “therapeutically effective amount” includes an amount or quantity effective, at dosages and for periods of time necessary, to produce a desired (e.g., therapeutic or prophylactic) result with respect to the indicated disease, disorder, or condition. The desired result may comprise a subjective or objective improvement in the recipient of the effective amount. In one non-limiting example, an effective amount of the compound of the present disclosure or a proline complex thereof includes an amount or dosage sufficient to decrease the blood pressure in an individual. The effective amount will vary with the type of subject being treated. Efficacy can also be expressed as “-fold” increase or decrease. For example, a therapeutically effective amount can have at least a 1.2-fold, 1.5-fold, 2-fold, 5-fold, or more effect over a control.

As used herein, the terms “subject”, “patient” or “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance. In some embodiments, a patient, subject or subject in need thereof is a human.

As used herein, “preselected” refers to the selection of one or more clinical, behavioral, and/or physiological criteria in a subject prior to initiating a therapeutic treatment. One or more clinical criteria can include the selection of conditions or diseases in a subject. One or more clinical criteria can also include the absence of one or more conditions or diseases in a subject. For example, a subject who is preselected to not have a particular disease was not diagnosed or was not exhibiting symptoms of said disease prior to initiating a therapeutic treatment. As an additional example, a subject who is preselected based on one or more behavioral criteria was practicing or exhibiting said behavioral criteria prior to initiating a therapeutic treatment.

As used herein, “blood pressure” refers to pressure exerted on the walls of blood vessels by blood that is pumped out of the heart and flows in the blood vessels. Generally, a subject's blood pressure is recorded as the systolic pressure (heart contraction) in mm Hg followed by the diastolic pressure (heart relaxation) in mm Hg (e.g. 120/88 mm Hg). The quantified pressures provided are usually the mean pressure over the course of multiple heart beats (i.e., more than one).

As used herein “resting blood pressure” refers to the blood pressure measured in a subject, as described above, where the subject has not recently engaged in moderate or vigorous physical activity. In some instances, “not recently” refers to at least 5, 10, 15, 20, 30, 60, or 90 minutes. Accordingly, “resting systolic blood pressure” and “resting diastolic blood pressure” refers to the resting form of each of systolic or diastolic blood pressures as described above.

As used herein, “primary hypertension” or “essential hypertension” refers to hypertension in patients without a medical cause that can be attributed to the hypertension. Primary hypertension may be promoted by a hereditary disposition, constitution (e.g. obesity), akinesia, or psychological strains. As used herein, in case there is a known cause for the hypertension such as an illness, damage or impairment of an organ, the hypertension is preferably referred to as “secondary hypertension”.

As used herein “pulse pressure” refers to the difference between the systolic and diastolic blood pressures.

As used herein “durable response” includes adequate relief of symptoms throughout the treatment regimen, and continuous adequate relief of symptoms throughout the treatment regimen. The duration of the durable response can be, for example, 2 weeks, 3 weeks, 4 weeks, 5 weeks, 6 weeks, 7 weeks, 8 weeks, 24 weeks, 48 weeks, 96 weeks or as long as the subject continues the treatment regimen.

### Methods

In one aspect, provided herein is a method of treating primary hypertension. The method includes administering to a subject in need thereof a therapeutically effective amount of Compound 1 ((2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol), having the formula:

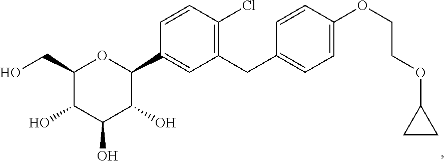 [SMILES: OC[C@H]1O[C@@H](c2ccc(Cl)c(Cc3ccc(OCCOC4CC4)cc3)c2)[C@H](O)[C@@H](O)[C@@H]1O]

or a pharmaceutically acceptable form thereof.

In some embodiments, Compound 1 is a bis-proline complex of (2S,3R,4R,5S,6R)-2-(4-chloro-3-(4-(2-cycl opropoxyethoxy)benzyl)phenyl)-6-(hydroxymethyl)tetrahydro-2H-pyran-3,4,5-triol, having the formula

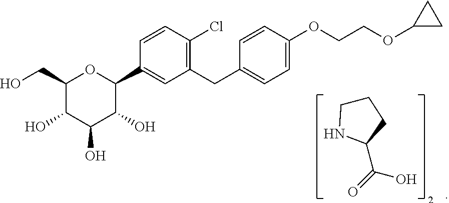

In some embodiments, Compound 1 is a crystalline form of (2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol, having the formula

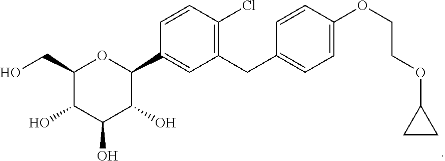 [SMILES: OC[C@H]1O[C@@H](c2ccc(Cl)c(Cc3ccc(OCCOC4CC4)cc3)c2)[C@H](O)[C@@H](O)[C@@H]1O]

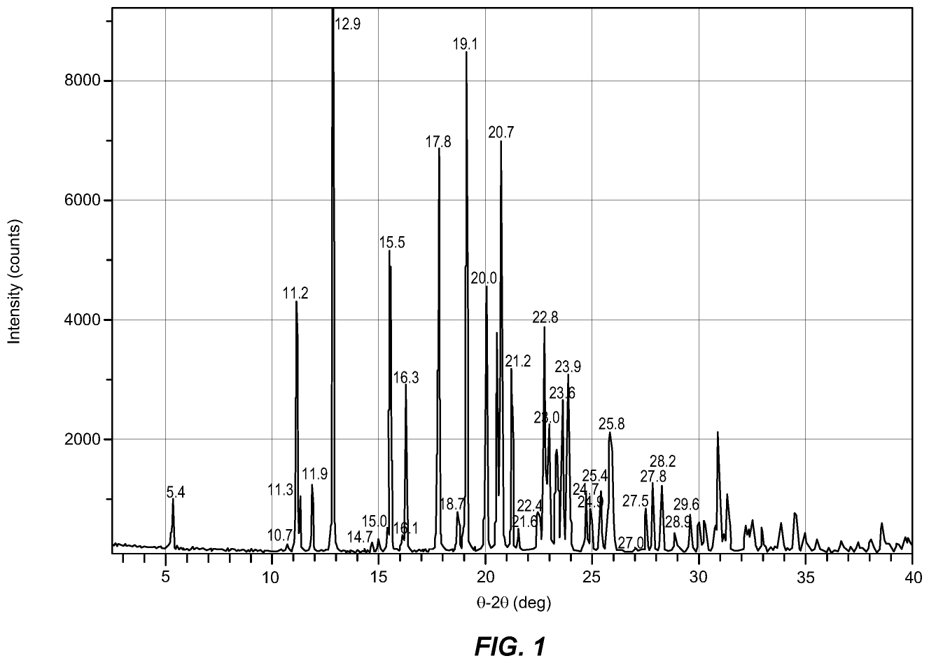

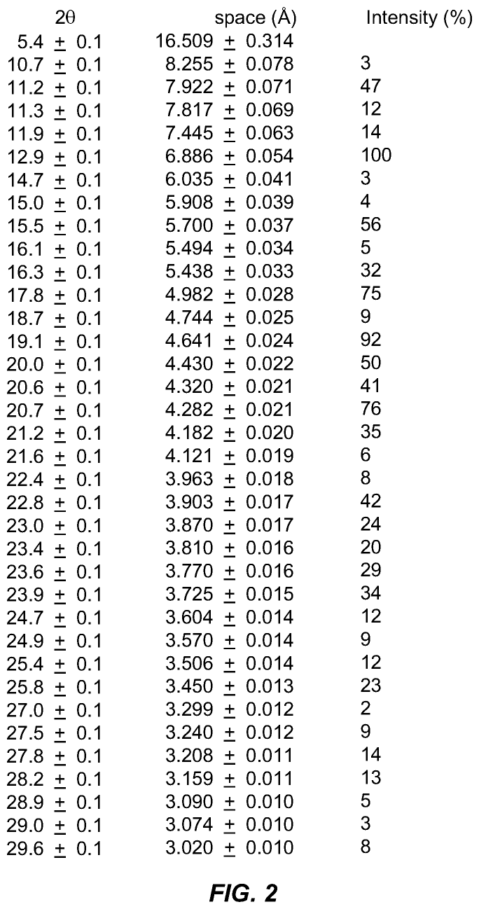

The crystalline form of the compound is characterized by an X-ray powder diffraction pattern shown in FIG. 1. In some embodiments, the X-ray powder diffraction pattern includes one or more peaks at 5.4, 11.2, 11.3, 11.9, 12.9, 15.5, 16.3, 17.8, 19.1, 20.0, 20.6, 20.7, 21.2, 22.8, 23.0, 23.4, 23.6, 23.9, 24.7, 25.4, 25.8, 27.8 and 28.2 degrees 2θ (±0.1 degrees 2θ), wherein said XRPD is made using CuK_{α1} radiation. In another embodiment, the crystalline form of the compound is characterized by an XRPD that includes two or more, three or more, four or more, or five or more peaks at 5.4, 11.2, 11.3, 11.9, 12.9, 15.5, 16.3, 17.8, 19.1, 20.0, 20.6, 20.7, 21.2, 22.8, 23.0, 23.4, 23.6, 23.9, 24.7, 25.4, 25.8, 27.8 and 28.2 degrees 2θ (±0.1 degrees 2θ). In some other embodiments, the crystalline form of the compound is characterized by an XRPD that includes peaks at 12.9, 19.1 and 20.7 degrees 2θ (±0.1 degrees 2θ). In still other embodiments, the crystalline form of the compound is characterized by an XRPD that includes peaks at 11.2, 12.9, 15.5, 17.8, 19.1, 20.0 and 20.7 degrees 2θ (±0.1 degrees 2θ). In yet other embodiments, the crystalline form of the compound is characterized by an XRPD that includes peaks at 5.4, 11.2, 11.9, 12.9, 15.5, 16.3, 17.8, and 19.1 degrees 2θ (±0.1 degrees 2θ). In still yet other embodiments, the crystalline form of the compound is characterized by an XRPD that includes peaks at 5.4, 11.2, 11.9, and 12.9 degrees 2θ (±0.1 degrees 2θ). In another embodiment, the crystalline form of the compound is characterized by an XRPD including peaks at 11.2 and 12.9 degrees 2θ (±0.1 degrees 2θ). In other embodiments, the crystalline form of the compound is characterized by the XRPD peaks substantially in accordance with FIG. 2.

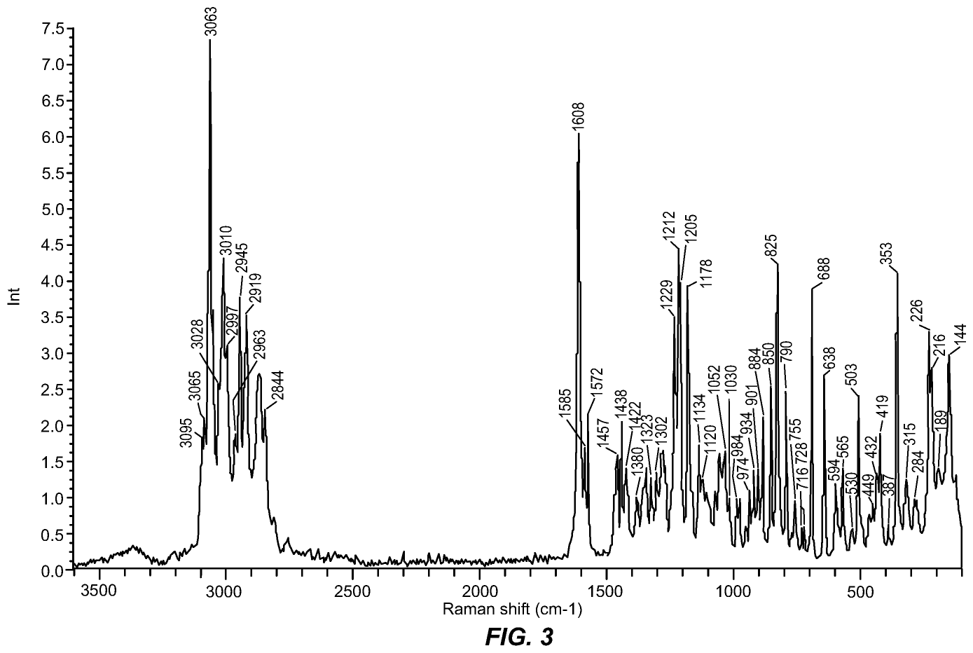

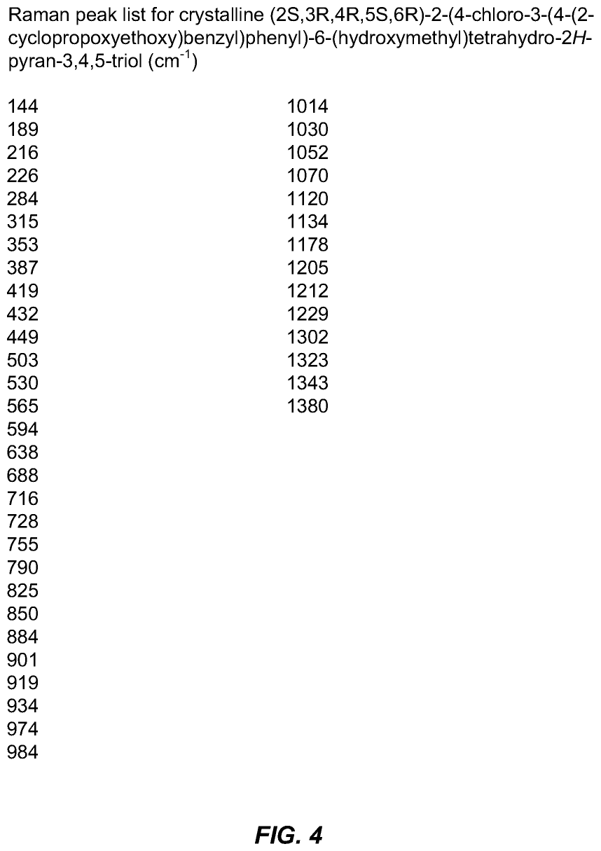

The crystalline compound of the present invention is also characterized by the Raman spectra substantially in accordance with FIG. 3 and the peaks substantially in accordance with FIG. 4. In some embodiments, the crystalline form of the compound is characterized by a Raman spectra that includes one or more peaks at about 353, 688, 825, 1178, 1205, 1212, 1608, 2945, 3010 and 3063 cm⁻¹. In another embodiment, the crystalline form of the compound is characterized by a Raman spectra that includes two or more, three or more, four or more, or five or more peaks. In other embodiments, the crystalline form of the compound is characterized by the Raman spectra including peaks at about 353, 688 and 825 cm⁻¹. In some other embodiments, the crystalline form of the compound is characterized by the Raman peaks substantially in accordance with FIG. 4.

In some embodiments, the therapeutically effective amount of Compound 1 is a total daily dosage of about 5 mg to 100 mg (e.g., about 5, 10, 15, 20, 25, 30, 35, 40, 45, 50, 55, 60, 65, 70, 75, 80, 85, 90, 85, or 100 mg/day). In some embodiments, the total daily dosage of Compound 1 is about 10 to 90 mg, 15 to 75 mg, 20 to 60 mg, 25 to 45 mg or 10 to 20 mg. In some embodiments, the total daily dosage of Compound 1 is about 10. In some embodiments, the total daily dosage of Compound 1 is about 20. In some embodiments, the total daily dosage of Compound 1 is about 30. In some embodiments, the total daily dosage of Compound 1 is about 40. In some embodiments, the total daily dosage of Compound 1 is about 50.

In some embodiments, Compound 1 is administered orally. In some embodiments, Compound 1 is administered daily in single, divided, or continuous doses. In some embodiments, Compound 1 is administered twice daily. In some embodiments, Compound 1 is administered three times daily.

In some embodiments, the subject receives daily dosages of Compound 1 for a period of from 1 to 36 weeks. In some embodiments, said subject receives daily dosages of Compound 1 for at least twelve weeks. In some embodiments, said subject receives daily dosages of Compound 1 for at least ten weeks. In some embodiments, said subject receives daily dosages of Compound 1 for at least eight weeks. In some embodiments, said subject receives daily dosages of Compound 1 for at least six weeks. In some embodiments, said subject receives daily dosages of Compound 1 for at least four weeks. In some embodiments, said subject receives daily dosages of Compound 1 for at least two weeks.

Administration of Compound 1 can provide In some embodiments, Compound 1 is administered daily for at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 or 84 days.

The treatment of hypertension by administration of Compound 1 occurs during the treatment regimen, but the administration of Compound 1 to a subject in need thereof can also provide a durable response in said subject. That is, said subject maintains adequate relief of symptoms after the cessation of the treatment regimen. For example, in some embodiments, said treating produces a durable response for at least 12 weeks. In some embodiments said treating produces a durable response for at least 24 weeks. In some embodiments, said treating produces a durable response for at least 48 weeks. In some embodiments, said treating produces a durable response for at least 96 weeks. In some embodiments, said treating produces a durable response for as long as Compound 1 is administered.

The subjects of the presently described treatment therapies may be preselected based one or more clinical, behavioral, and/or physiological criteria. In some embodiments, the subject is preselected to be obese. In some embodiments, the subject is preselected to not be obese. Obese is defined as having a body mass index (BMI) of 30 and above. In some embodiments, the subject is preselected to smoke at least 5 cigarettes a week. In some embodiments, the subject is preselected to not smoke. In some embodiments, the subject is preselected to consume at least 7 alcoholic beverages a week. In some embodiments, the subjects are preselected based on the presence or absence of particular conditions or disease states. In some embodiments, the subject is preselected to not have type 1 or 2 diabetes. In some embodiments, the subject is preselected to not have type 2 diabetes.

In some embodiments, the subject is preselected to be pregnant. In some embodiments, the subject is preselected to have preeclampsia.

The treatment therapies described herein are generally suitable for subjects with primary hypertension. This can include subjects with a high normal blood pressure or a hypertensive blood pressure. High normal blood pressure means a resting systolic blood pressure of about 130-139 mm Hg and/or a resting diastolic blood pressure of about 85-89 mm Hg. Hypertensive blood pressures can be broken into three stages: Stage 1, Stage 2, or Stage 3. Stage 1 hypertension means a resting systolic blood pressure of about 140-159 mm Hg and/or a resting diastolic blood pressure of about 90-99 mm Hg. Stage 2 hypertension means a resting systolic blood pressure of about 160-179 mm Hg and/or a resting diastolic blood pressure of about 100-109 mm Hg. Stage 3 hypertension means a resting systolic blood pressure of ≥180 mm Hg and/or a resting diastolic blood pressure of ≥110 mm Hg. In some embodiments, said subject has an initial blood pressure, prior to treatment, of about 140/90 mm Hg or higher.

In some embodiments a subject in need thereof is diagnosed with high normal blood pressure. In some embodiments, a subject in need thereof is diagnosed with Stage 1, Stage 2, or Stage 3 hypertension. In some embodiments, a subject in need thereof is diagnosed with Stage 1, hypertension. In some embodiments, a subject in need thereof is diagnosed with Stage 2 hypertension. In some embodiments, a subject in need thereof is diagnosed with Stage 3 hypertension.

The therapeutic treatment achieved will vary from subject to subject and depend on factors including, but not limited to, initial blood pressure, amount of Compound 1 administered, and the duration of treatment. The treatments presently disclosed will reduce the systolic and/or the diastolic blood pressure of the subjects.

In some embodiments, said treating reduces resting systolic blood pressure. In some embodiments, said treating reduces resting systolic blood pressure in said subject by about 3 to 20 mm Hg. In some embodiments, said treating reduces resting systolic blood pressure in said subject by at least 3 mm Hg. In some embodiments, said treating reduces resting systolic blood pressure in said subject by at least 5 mm Hg. In some embodiments, said treating reduces resting systolic blood pressure in said subject by at least 7 mm Hg. In some embodiments, said treating reduces resting systolic blood pressure in said subject by at least 10 mm Hg. In some embodiments, said treating reduces resting systolic blood pressure in said subject by at least 15 mm Hg. In some embodiments, the resting systolic blood pressure is the seated office systolic blood pressure of the subject.

In some embodiments, said treating reduces resting diastolic blood pressure. In some embodiments, said treating reduces resting diastolic blood pressure in said subject by about 2 to 15 mm Hg. In some embodiments, said treating reduces resting diastolic blood pressure in said subject by at least 2 mm Hg. In some embodiments, said treating reduces resting diastolic blood pressure in said subject by at least 4 mm Hg. In some embodiments, said treating reduces resting diastolic blood pressure in said subject by at least 6 mm Hg. In some embodiments, said treating reduces resting diastolic blood pressure in said subject by at least 8 mm Hg. In some embodiments, said treating reduces resting diastolic blood pressure in said subject by at least 10 mm Hg. In some embodiments, the resting diastolic blood pressure is the seated office diastolic blood pressure of the subject.

In some embodiments said treating reduces the pulse pressure, that is, the difference between the systolic and diastolic blood pressures, in said subject by about 2 to 15 mm Hg. In some embodiments, said treating reduces resting pulse pressure in said subject by at least 2 mm Hg. In some embodiments, said treating reduces resting pulse pressure in said subject by at least 5 mm Hg. In some embodiments, said treating reduces resting pulse pressure in said subject by at least 7 mm Hg. In some embodiments, said treating reduces resting pulse blood pressure in said subject by at least 10 mm Hg.

### Combination Therapy

In other aspects of the present invention, therapies that include Compound 1 (or a pharmaceutically acceptable form thereof), will be used in combination with other anti-hypertensive agents. Agents to be used in combination with Compound 1, for example, include: vasodilators such as angiotensin-converting enzyme (ACE) inhibitors or angiotensin II receptor blockers (ARBs); calcium channel blockers, alpha adrenergic receptor blockers, beta adrenergic receptor blockers (beta blockers), and thiazide diuretics or loop diuretics.

In some embodiments, the administration of Compound 1 reduces the dosage or frequency of dosing of other antihypertensive medications.

In some embodiments, the administration of Compound 1 decreases the frequency of hypotensive episodes in subjects experiencing fluctuations in their daily blood pressure. In some embodiments, the administration of Compound 1 reduces the severity of hypotensive episodes in subjects experiencing such episodes.

In some embodiments, the administration of Compound 1 prevents the appearance of diabetes in subjects exposed to antihypertensive agents that increase the risk of diabetes, such as thiazide diuretics or beta blockers.

### Pharmaceutical Compositions

Compound 1 can be prepared in various compositions suitable for delivery to a subject. A composition suitable for administration to a subject typically comprises Compound 1, (or a pharmaceutically acceptable form thereof and a pharmaceutically acceptable carrier.

Compound 1 can be incorporated into a variety of formulations for therapeutic administration. More particularly, Compound 1 can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of a compound of the present invention can be achieved in various ways, including oral, buccal, parenteral, intravenous, intradermal (e.g., subcutaneous, intramuscular), transdermal, etc., administration. Moreover, the Compound 1 can be administered in a local rather than systemic manner, for example, in a depot or sustained release formulation.

The pharmaceutical compositions for the administration of Compound 1 can conveniently be presented in unit dosage form and can be prepared by any of the methods known in the art of pharmacy and drug delivery. All methods include the step of bringing the active ingredient into association with a carrier containing one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active agent is generally included in an amount sufficient to produce a decrease in a subject's blood pressure.

Suitable formulations for use in the present invention are found in Remington: THE SCIENCE AND PRACTICE OF PHARMACY, 21st Ed., Gennaro, Ed., Lippincott Williams & Wilkins (2003), which is hereby incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

In some embodiments, Compound 1 is prepared for delivery in a sustained-release, controlled release, extended-release, timed-release or delayed-release formulation, for example, in semipermeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide, zinc oxide, and clay (see, U.S. Pat. No. 6,638,521, herein incorporated by reference). Exemplified extended release formulations that can be used in delivering Compound 1 (in any of the forms described herein) include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Controlled release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

For oral administration, Compound 1 can be readily formulated by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Tablets of the current disclosure contain the active ingredient in admixture with non-toxic pharmaceutically acceptable excipients which are suitable for the manufacture of tablets. These excipients may be, for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for controlled release.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

In some instances, Compound 1 can be formulated for parenteral administration by injection, e.g., by bolus injection or continuous infusion. For injection, the compound can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, Compound 1 can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain agents such as suspending, stabilizing and/or dispersing agents.

Pharmaceutical formulations for parenteral administration include aqueous solutions of Compound 1 (in any of the forms noted herein) in water-soluble form. Additionally, suspensions of Compound 1 can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions. Alternatively, Compound 1 can be in powder form for constitution with a suitable vehicle, e.g., sterile pyrogen-free water, before use.

Systemic administration can also be by transmucosal or transdermal means. For transmucosal or transdermal administration, penetrants appropriate to the barrier to be permeated are used in the formulation. For topical administration, Compound 1 can be formulated into ointments, creams, salves, powders and gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplified transdermal delivery formulations that can find use in the present invention include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

For buccal administration, the compositions can take the form of tablets or lozenges formulated in conventional manner.

In addition to the formulations described previously, Compound 1 can also be formulated as a depot preparation. Such long acting formulations can be administered by implantation (for example subcutaneously or intramuscularly) or by intramuscular injection. Thus, for example, Compound 1 can be formulated with suitable polymeric or hydrophobic materials (for example as an emulsion in an acceptable oil) or ion exchange resins, or as sparingly soluble derivatives, for example, as a sparingly soluble complex or salt.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions wherein Compound 1 is contained in a therapeutically effective amount. The present invention also contemplates pharmaceutical compositions comprising Compound 1 (in any of the forms noted herein) in admixture with an effective amount of other therapeutic agents as combination partners, particularly those used for treating diseases and conditions which can be affected by SGLT inhibition, such as antidiabetic agents, lipid-lowering/lipid-modulating agents, agents for treating diabetic complications, anti-obesity agents, antihypertensive agents, antihyperuricemic agents, and agents for treating chronic heart failure, atherosclerosis or related disorders. An effective amount of the compound and/or combination partner will, of course, be dependent on the subject being treated, the severity of the affliction and the manner of administration. Determination of an effective amount is well within the capability of those skilled in the art, especially in light of the detailed disclosure provided herein. Generally, an efficacious or effective amount of a compound is determined by first administering a low dose or small amount, and then incrementally increasing the administered dose or dosages until a desired therapeutic effect is observed in the treated subject, with minimal or no toxic side effects. Applicable methods for determining an appropriate dose and dosing schedule for administration of the present invention are described, for example, in GOODMAN AND GILMAN'S THE PHARMACOLOGICAL BASIS OF THERAPEUTICS, 11th Ed., Brunton, Lazo and Parker, Eds., McGraw-Hill (2006), and in REMINGTON: THE SCIENCE AND PRACTICE OF PHARMACY, 21st Ed., Gennaro, Ed., Lippincott Williams & Wilkins (2003), both of which are hereby incorporated herein by reference.

### Pharmaceutical Dosage Forms

The present disclosure includes novel pharmaceutical dosage forms of Compound 1, or a pharmaceutically acceptable form thereof. The dosage forms described herein are suitable for oral administration to a subject. The dosage form may be in any form suitable for oral administration, including, but not limited to, a capsule or a tablet.

In some embodiments, the present disclosure provides a single unit dosage capsule or tablet form containing 5-100 mg of Compound 1, having the formula:

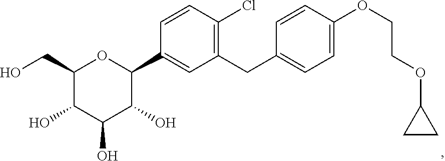 [SMILES: OC[C@H]1O[C@@H](c2ccc(Cl)c(Cc3ccc(OCCOC4CC4)cc3)c2)[C@H](O)[C@@H](O)[C@@H]1O]

or a proline complex or crystalline form thereof.

In some embodiments, the amount of Compound 1 is from about 10 to 90 mg. In some embodiments, the amount of Compound 1 is from about 15 to 75 mg. In some embodiments, the amount of Compound 1 is from about 20 to 60 mg. In some embodiments, the amount of Compound 1 is from about 25 to 45 mg. In some embodiments, the amount of Compound 1 is from about 10 to 20 mg. In some embodiments, the amount of Compound 1 is about 5, 10, 15, 20, 25, 30, 35, 40, 45, 50, 55, 60, 65, 70, 75, 80, 85, 90, 95, 100 mg. In some embodiments, the amount of Compound 1 is about 10 mg. In some embodiments, the amount of Compound 1 is about 20 mg. In some embodiments, the amount of Compound 1 is about 25 mg. In some embodiments, the amount of Compound 1 is about 50 mg.

In some embodiments, the single unit dosage form of Compound 1 is a capsule. In some embodiments, the single unit dosage form of Compound 1 is a tablet.

In some embodiments, the single unit dosage form is in a capsule of size #0, #1, #2, #3, #4, or #5. In some embodiments, the single unit dosage form is in a capsule of size #4. In some embodiments, the single unit dosage form is in a capsule of size #5.

### Kits

Also provided herein are kits comprising pharmaceutical compositions and dosage forms of Compound 1, or forms thereof, and including kits for combination therapy.

In some aspects, the present invention provides a kit that includes Compound 1. Some of the kits described herein include a label describing a method of administering Compound 1. Some of the kits described herein include a label describing a method of treating primary hypertension. In some embodiments, the kits described herein include a label describing a method of reducing a subject's blood pressure.

The compositions of the present invention, including but not limited to, compositions comprising Compound 1 in a bottle, jar, vial, ampoule, tube, or other container-closure system approved by the United States Food and Drug Administration (FDA) or other regulatory body, which may provide one or more dosages containing the compounds. The package or dispenser may also be accompanied by a notice associated with the container in a form prescribed by a governmental agency regulating the manufacture, use, or sale of pharmaceuticals, the notice indicating approval by the agency. In certain aspects, the kit may include a formulation or composition as described herein, a container closure system including the formulation or a dosage unit form including the formulation, and a notice or instructions describing a method of use as described herein.

All publications and patent applications cited in this specification are herein incorporated by reference as if each individual publication or patent application were specifically and individually indicated to be incorporated by reference. Any conflict between any reference cited herein and the teaching of this specification is to be resolved in favor of the latter. Similarly, any conflict between an art-recognized definition of a word or phrase and a definition of the word or phrase as provided in this specification is to be resolved in favor of the latter.

Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims. The invention will be described in greater detail by way of specific examples.

### EXAMPLES

The following examples are offered for illustrative purposes, and are not intended to limit the invention in any manner. Those of skill in the art will readily recognize a variety of noncritical parameters which can be changed or modified to yield essentially the same results.

### Example 1: Double-Blind Placebo-Controlled Trial

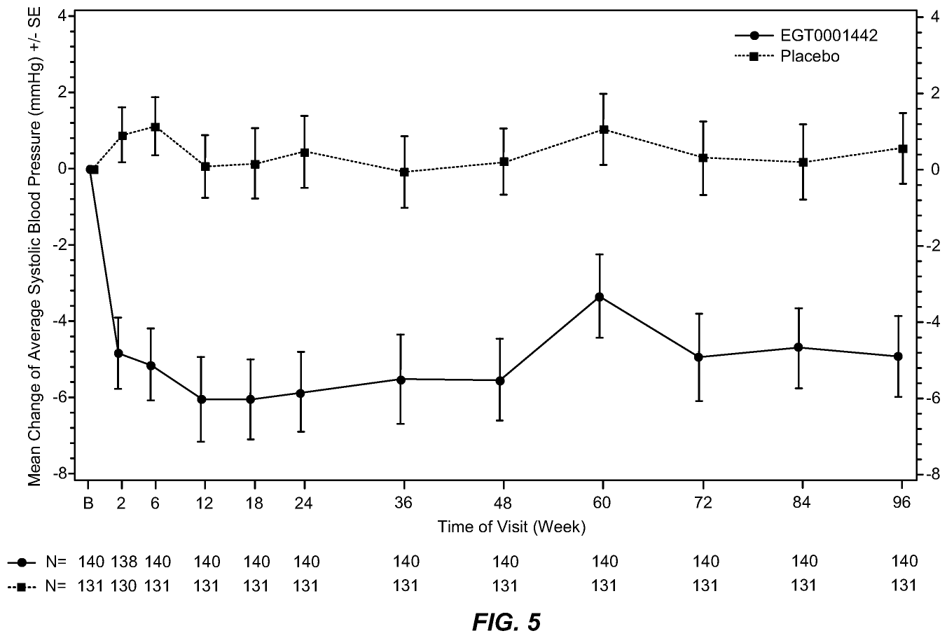

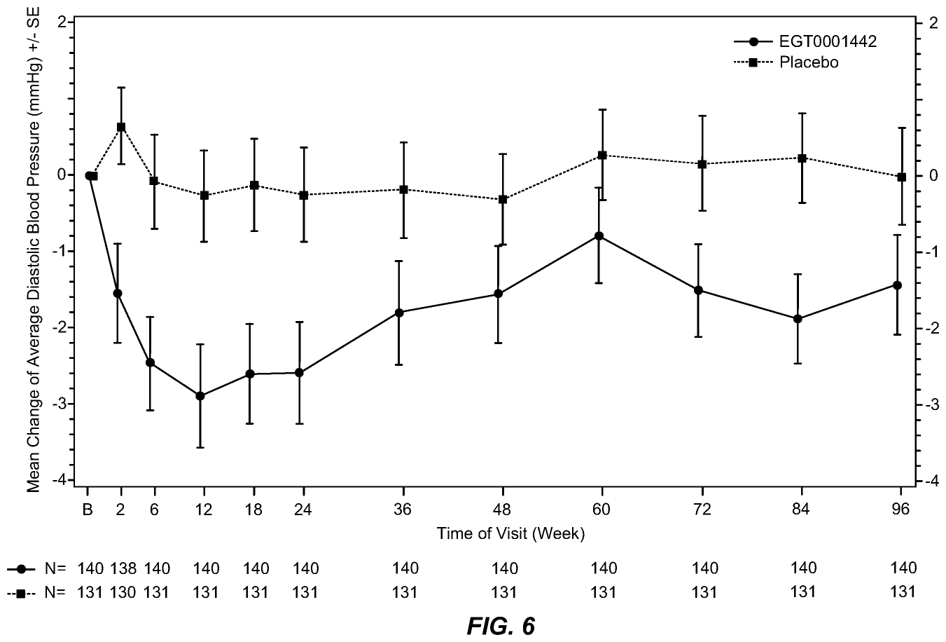

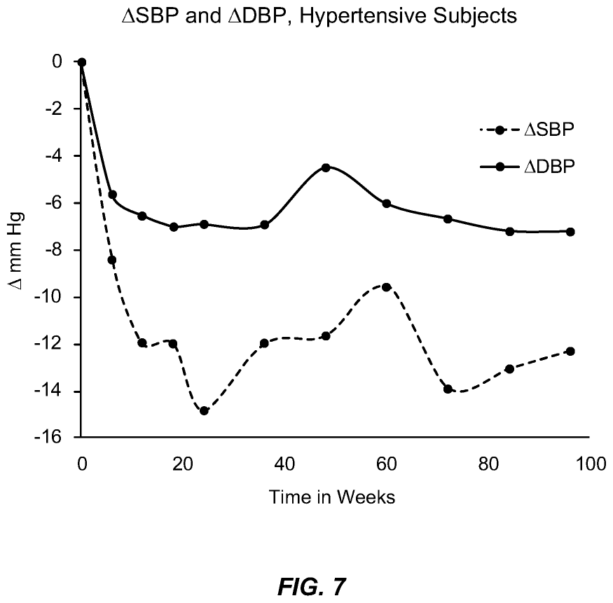

In a multicenter double-blind placebo-controlled trial, subjects with diabetes were administered Compound 1, 20 mg per day or placebo for 96 weeks. The blood pressure of resting seated subjects was measured at every office visit. Although the study population was not specifically recruited to contain participants with hypertension, a fraction of the population was affected by the disease and the mean baseline systolic blood pressure of the entire study population was 127 mm Hg. The effects of Compound 1 and placebo on systolic and diastolic blood pressure as a function of time are shown in FIG. 5 (systolic) and FIG. 6 (diastolic). The effects of Compound 1 on the change in systolic and diastolic blood pressure as a function of time among subjects who had a systolic blood pressure of 140 mm Hg or above at study entry is shown in FIG. 7. An approximately 7 mm Hg reduction in diastolic blood pressure and an approximately 12 mm Hg reduction in systolic blood pressure developed and was found to persist for the duration of the trial.

### Systolic Blood Pressure

The mean change from baseline of the systolic blood pressure in the Compound 1 group in the study of Example 1 was a decrease of approximately −3 mm Hg to −6 mm Hg that persisted throughout the treatment period. The mean change from baseline of the systolic blood pressure in the placebo group throughout the treatment period was small.

In the Compound 1 group, mean [SD] changes from baseline were −4.80 [10.875] mm Hg at Week 2, −5.84 [12.707] mm Hg at Week 24, and −4.90 [12.513] mm Hg at Week 96; corresponding values in the placebo group were 0.91 [8.316] mm Hg, 0.46 [10.915] mm Hg, and 0.55 [10.591] mm Hg.

At all other visits (Weeks 6, 12, 18, 36, 48, 60, 72, and 84), mean decreases in the Compound 1 group ranged from −3.32 mm Hg to −6.03 mm Hg; in contrast, in the placebo group, values ranged from −0.07 mm Hg to 1.11 mm Hg.

A 2-sample t-test showed the differences in the change from baseline between treatment groups were statistically significant at Week 2 (p<0.0001), Week 24 (p<0.0001), and Week 96 (p=0.0001) and at all other visits (p<0.0031).

### Diastolic Blood Pressure

The mean change from baseline of the diastolic blood pressure in the compound 1 group of the study of Example 1 was a decrease of approximately −0.80 mm Hg to −3 mm Hg that persisted throughout the treatment period. The mean change from baseline of the diastolic blood pressure in the placebo group was small throughout the treatment period.

In the Compound 1 group, mean [SD] changes from baseline were −1.55 [7.538] mm Hg at Week 2, −2.59 [7.898] mm Hg at Week 24, and −1.43 [7.711] mm Hg at Week 96; in the placebo group, the corresponding values were 0.65 [5.797] mm Hg, −0.25 [7.125] mm Hg, and −0.01 [7.217] mm Hg, respectively. At all other visits, the mean changes in the Compound 1 group ranged from −0.79 mm Hg to −2.89 mm Hg. For the placebo group, no notable pattern was observed; there were increases in diastolic blood pressure at some visits and decreases at others.

The differences between treatment groups, as shown by a 2-sample t-test, were statistically significant at Week 2 (p=0.0079) and Week 24 (p=0.0113), as well as at Weeks 6, 12, 18, and 84 (all p-values were <0.0128); however, the difference between treatment groups approached but did not reach statistical significance at Week 72 (p=0.0576), and there were no statistically significant differences between treatment groups at Weeks 36, 48, 60, and 96.

### Example 2: Safety Events Related to Blood Pressure

A treatment emergent adverse event (TEAE) is an adverse event that develops during or immediately following the period of exposure to the investigational product in a clinical trial. An adverse event is any event that represents an unfavorable change in the health of the individual that is clinically significant—it may represent a new condition or status or materially worsened pre-existing condition. In the clinical trial of Example 1, hypertension was recorded as a TEAE in 2 subjects that had been administered Compound 1 and in 5 subjects administered placebo. A hypertensive crisis was observed in 0 subjects administered Compound 1 and in 3 subjects administered placebo. One episode of hypotension was observed among subjects administered Compound 1 and none among subjects administered placebo. The lower incidence of hypertensive TEAEs and the single episode of hypotension are consistent with the antihypertensive effect of Compound 1. In addition, the observation of only a single episode of hypotension is notable because the mean systolic blood pressure at baseline was only 127 mm Hg, well below the 140 mm Hg level that demarks a widely adopted standard for the diagnosis of hypertension. Thus, Compound 1 has a favorably low propensity for producing deleterious reductions in blood pressure.

### Example 3: Double-Blind, Placebo-Controlled Trial in a Population not Pre-Selected for Diabetes

Study THR-1442-C-603A randomized to bexagliflozin tablets, 20 mg, or to bexagliflozin tablets, placebo, subjects who exhibited a seated office systolic blood pressure of ≥140 mm Hg and <180 mm Hg, who were receiving no more than 4 anti-hypertensive medications, and who qualified for participation by demonstrating the ability to faithfully self-administer medication and to produce a qualified 24 h ambulatory blood pressure monitoring (ABPM) record. A qualified ABPM record contained no fewer than 64 measurements over an approximately 24 h period and a mean SBP≥135 mm Hg.

Potential participants were excluded if they had type 1 diabetes, secondary or malignant hypertension, an office seated DBP>110 mm Hg, if they had been prescribed insulin or more than 4 anti-hypertensive medications, had a predisposition to urinary tract infections, a history of cancer within the previous 3 years, of alcohol or drug abuse in the previous 2 years, of stroke, myocardial infarct or hospitalization for heart failure in the previous 6 months, of SGLT2 exposure in the previous 3 months, had a kidney transplant, an estimated glomerular filtration rate less than 45 mL/min/1.73 m2, an HbA1c greater than 9.5%, or physical or occupational factors precluding ABPM measurement.

A total of 678 subjects were randomly assigned to the active arm or to the placebo arm. Randomization was stratified to ensure that approximately equal numbers of subjects in each arm had a history of diabetes or not, were presently medicated for hypertension or not, had intact renal function (as determined by an estimated glomerular filtration rate of ≥60 mL min⁻¹ per 1.73 m² of body surface area) or not, and had more advanced disease (ABPM SBP≥160 mm Hg) or not.

The study was conducted in the United States at multiple clinical sites. The randomized population consisted of 409 male and 269 female participants with a mean age ±standard deviation of 58.5±11.74. 33.2% of subjects had an age ≥65 years. 64.7% were self-declared Caucasian, 29.9% African-American, 1.6% Asian, 0.3% Native American/Native Alaskan, 0.1% Native Hawaiian/Other Pacific Islander, 1.0% Multiracial and 2.2% Other. 8.1% self-declared Hispanic or Latino ethnicity. The study population was obese, having a median body mass index (kg m⁻²) of 31.9 and a mean weight of 98.3±35.0 kg. A history of diagnosed diabetes was elicited from 24.3% of the subjects and 18.0% were presently unmedicated for hypertension. 7.5% of the subjects exhibited signs of renal impairment.

The baseline 24 h average SBP from ABPM was 147.3±9.93 and the DBP was 84.5±9.65. 11.7% of the subjects produced a baseline ABPM SBP≥160 mm Hg.

Following self-administration of investigational product for 12 weeks, subjects underwent ABPM to assess the potential treatment effect. After the first 300 subjects had completed 12 weeks of participation, the mean SBP was 140.90, corresponding to a decrease from baseline in the entire population of 6.4 mm Hg.

### Example 4: Full Analysis of a Double-Blind, Placebo-Controlled Trial in a Population not Pre-Selected for Diabetes

A total of 678 subjects were randomized, of whom 5 were excluded for major protocol violations. Of the remaining 673 participants, 307 out of 334 in the bexagliflozin arm and 319 out of 339 in the placebo arm completed the study. The arms were well-balanced in general by demographic characteristics. The population was predominantly male (60.5%) and Caucasian (65.2%) with the remainder by race predominantly of African heritage (29.4%). Participants of Latino ethnicity comprised 8.0% of the total. The mean age was 58.9 years and 24.8% were diabetic. The mean subject was obese with a BMI of 32.6 kg/m², and 81% reported at least one prescription for anti-hypertensive medication. ACE inhibitors and ARBs were the most commonly encountered agents. Calcium channel blockers, diuretics and β-blockers were the next most commonly encountered, in that order. The mean seated office blood pressure was 156/91 with a heart rate of 73. The mean ABPM at baseline was 147.5/85.0. Nearly all participants had preserved renal function.

A large placebo effect was detected in the study, but the placebo-adjusted change in seated office SBP from baseline to the study conclusion at 12 weeks was −4.63 mm Hg, (95% confidence interval −6.83, −2.42; p<0.0001). The placebo-adjusted change in seated office DBP from baseline to week 12 was −2.31 mm Hg, (95% CI −3.70, −0.91, p=0.0011). The proportion of subjects achieving a seated office SBP≤140 mm Hg was 0.38 in the bexagliflozin arm compared to 0.25 in the placebo arm, with an odds ratio for achievement of the desired outcome of 1.86 (95% CI 1.32, 2.62, p=0.0004). The proportion of subjects achieving a seated office DBP≤90 mm Hg was 0.65 in the bexagliflozin arm compared to 0.53 in the placebo arm, with an odds ratio for achievement of the desired outcome of 1.63 (95% CI 1.13, 2.35, p=0.0094).

An analysis of hourly change in blood pressure by ambulatory blood pressure monitoring showed that the anti-hypertensive effect did not show significant waning over the 24 h data collection period. Analysis of the ABPM data also demonstrated a smaller treatment effect than observed by seated office measurements. A placebo-adjusted reduction in ambulatory mean SBP from baseline to week 12 of 2.72 mm Hg (p=0.0025) was detected among participants in the bexagliflozin arm and the mean placebo-adjusted change from baseline to week 12 was −0.83 mm Hg (p=0.0863) by ambulatory measurement. Among the subgroups analyzed by ABPM SBP measurement, the largest treatment effects were for participants with stage 3 CKD (−7.53 mm Hg, p=0.0571), concomitant β-blocker use (−6.26 mm Hg, p=0.0012), a history of diabetes (−6.07 mm Hg, p=0.0023), concomitant diuretic use (−5.60 mm Hg, p=0.0021) and use of 3 or more anti-hypertensive medications (−5.55 mm Hg, p=0.0071).

The distribution and overall number of adverse events was generally balanced between the two arms. There were slightly more adverse events by percentage among participants in the placebo cohort (46.6% compared to 41.9%) and serious adverse events were balanced between the arms. There was an excess of adverse events leading to subject withdrawal or treatment discontinuation in the active arm (1.8% compared to 0.9%), with the excess accounted for by objectionable increased frequency of urination and micturition urgency.

Overall, older subjects, subjects with a history of diabetes at baseline, and subjects reporting 3 or more anti-hypertensive medications showed greater responses to bexagliflozin. A large effect was observed among subjects reporting β-blocker use, who responded less well to the placebo product. The treatment effect was also greater among subjects who experienced a nocturnal decline in blood pressure. Although the sample size was small, participants with stage 3 CKD at screening responded well to bexagliflozin.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of treating primary hypertension in a subject in need thereof, said method comprising administering to said subject a therapeutically effective amount of Compound 1, having the formula: 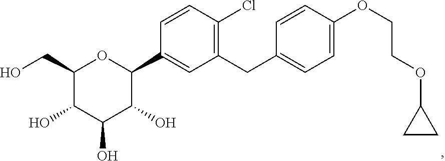 [SMILES: OC[C@H]1O[C@@H](c2ccc(Cl)c(Cc3ccc(OCCOC4CC4)cc3)c2)[C@H](O)[C@@H](O)[C@@H]1O]
or a pharmaceutically acceptable form thereof.

2. The method of claim 1, wherein Compound 1 is a bis-proline complex of (2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol, having the formula

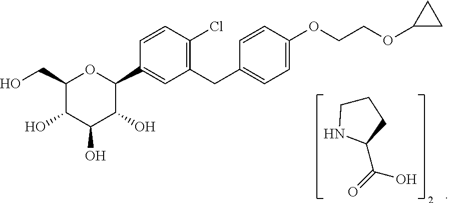

3. The method of claim 1, wherein Compound 1 is a crystalline form of (2S,3R,4R,5 S,6R)-2-(4-chloro-3-(4-(2-cyclopropoxyethoxy)benzyl)phenyl)-6-(hydroxymethyl)tetrahydro-2H-pyran-3,4,5-triol, having the formula

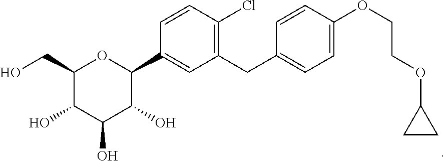 [SMILES: OC[C@H]1O[C@@H](c2ccc(Cl)c(Cc3ccc(OCCOC4CC4)cc3)c2)[C@H](O)[C@@H](O)[C@@H]1O]

4. A method of claim 1, wherein said therapeutically effective amount of Compound 1 is a total daily dosage of from about 5 to 100 mg per day.

5.-14. (canceled)

15. The method of claim 1, wherein said subject receives daily dosages of Compound 1 for at least twelve weeks.

16. The method of claim 1, wherein said subject is preselected to not have type 2 diabetes.

17.-21. (canceled)

22. The method of claim 1, wherein said subject is preselected to have preeclampsia.

23. The method of claim 1, wherein said subject has a high normal blood pressure or a hypertensive blood pressure.

24. The method of claim 23, wherein said subject has an initial blood pressure, prior to treatment, of about 140/90 mm Hg or higher.

25. The method of claim 1, wherein said treating reduces resting systolic blood pressure in said subject by about 3 to 20 mm Hg.

26.-30. (canceled)

31. The method of claim 25, wherein said resting systolic blood pressure is the seated office systolic blood pressure of said subject.

32. The method of claim 1, wherein said treating reduces resting diastolic blood pressure in said subject by about 2 to 15 mm Hg.

33.-36. (canceled)

37. The method of claim 32, wherein said resting diastolic blood pressure is the seated office diastolic blood pressure of said subject.

38. The method of claim 1, wherein said treating reduces the pulse pressure in said subject by about 2 to 15 mm Hg.

39.-41. (canceled)

42. The method of claim 38, further comprising administration of another anti-hypertensive agent.

43. (canceled)

44. The method of claim 42, wherein administration of Compound 1 reduces the dosage or frequency of dosing of said anti-hypertensive agent when the agent is used alone.

45. The method of claim 42, wherein a reduction in severity or frequency of hypotensive episodes is experienced in subjects initially having fluctuations in their daily blood pressure.

46. The method of claim 1, wherein Compound 1 is in the form of a pharmaceutical composition comprising about 5-100 mg of Compound 1.

47. (canceled)

48. (canceled)

49. The method of claim 1, wherein Compound 1 is in the form of a single unit dosage capsule comprising about 5-100 mg of Compound 1.

50. (canceled)

51. (canceled)

52. The method of claim 1, wherein Compound 1 is in the form of a single unit dosage tablet comprising about 5-100 mg of Compound 1.

53.-56. (canceled)
